(19) United States
(12) Patent Application Publication
(10) Pub. No.: US 2021/0330686 A1
(43) Pub. Date: Oct. 28, 2021

(54) COMPOSITION CONTAINING 2'-FUCOSYLLACTOSE FOR PREVENTING STROKE

(71) Applicant: Advanced Protein Technologies Corp., Suwon-si, Gyeonggi-do (KR)

(72) Inventors: Chul Soo SHIN, Suwon-si, Gyeonggi-do (KR); Jong Won YOON, Seongnam-si, Gyeonggi-do (KR); Chang Ku JEONG, Suwon-si, Gyeonggi-do (KR); Young Ha SONG, Yongin-si, Gyeonggi-do (KR); Seong Jin YU, Zhunan, Miaoli (TW)

(73) Assignee: Advanced Protein Technologies Corp., Suwon-si, Gyeonggi-do (KR)

(21) Appl. No.: 16/613,665

(22) PCT Filed: May 15, 2019
(86) PCT No.: PCT/KR2019/005812

§ 371 Date: Nov. 14, 2019

(30) Foreign Application Priority Data
May 15, 2018 (KR) 10-2018-0055182

(51) Int. Cl. A61K 31/702; A61P 9/10

(52) U.S. Cl. CPC A61K 31/702; A61P 9/10

(57) ABSTRACT

Disclosed is a food or pharmaceutical composition containing 2′-fucosyllactose (2′-FL) for preventing a stroke. The food or pharmaceutical composition containing 2′-fucosyllactose (2′-FL) is highly effective in preventing a stroke.

### TECHNICAL FIELD

The present invention relates to a food or pharmaceutical composition containing 2′-fucosyllactose (2′-FL) for preventing a stroke.

### BACKGROUND ART

The human brain is an important organ responsible for high-level mental activities such as intelligence, judgment, memory and thinking skills, which is the basis for human life and culture, which distinguishes humans from animals. The number of neurons that constitute the human brain is estimated to be between 10 billion and 100 billion, and it is thought that one lives one's entire life with the neurons present at birth, because they do not regenerate. In addition, about 30 to 200,000 neurons are known to continue to die every day after age 30. The brain is a very sensitive tissue that should receive about 30% of the systolic blood volume in order to maintain normal activity. Loss of consciousness occurs when blood supply is blocked even for only 20 seconds, and when the loss of consciousness lasts for 4 to 8 minutes, the brain cells of the central nervous system undergo irreversible changes and then die.

Stroke, which is caused by ischemia, is a disease with high mortality, along with cancer and heart diseases. Dementia patients in Korea mostly suffer from vascular dementia, but early detection and treatment are important to prevent fatal dementia or stop the progression of dementia. Vascular dementia is dementia caused by cerebrovascular disease and 8 to 35% of dementia patients suffer from vascular dementia. Most patients with vascular dementia survive for about 5 to 6 years after the onset of dementia, and mainly die from cardiovascular disease or a stroke. Adult dementia can be defined as a disease caused by acquired impairment of important mental activities such as language, memory, spatial and temporal relations, emotions, personality, and cognition of patients. When blood supply to a specific part of the brain parenchyma is interrupted due to blood vessel occlusion or bleeding, the supply of oxygen-glucose to nervous tissue is stopped.

This causes dysfunction of ATP-dependent ion channels present in the cell membrane. As a result, a large amount of cations such as calcium cations are introduced through the cell membrane to depolarize the nerve cells, increase the release of neurotransmitters including glutamate at the nerve ends, reduce the re-absorption of glutamate by the glial cells and allow glutamate, an excitatory neuronal substance, to accumulate at nerve junctions. Excessive accumulation of glutamate leads to the death of neurons due to the activity of N-methyl-D-aspartate (NMDA), AMPA, and kainate receptors. Based on these reports, research is underway on the effects of protecting brain cells by ischemia using NMDA inhibitors, Na⁺, Ca²⁺-channel inhibitors, glutamate inhibitors, GABA stimulators or the like.

Refractory neurological diseases such as stroke or neurodegenerative diseases commonly result in the death of brain cells or nerve cells, causing irreversible higher neurological dysfunction. Currently, treatment using various kinds of compounds and stem cells has been attempted using animal models all over the world, but there are many problems associated with application thereof to clinical trials. Therefore, there is a need to consider methods capable of effectively protecting brain or nerve cells prior to death of these cells.

### DISCLOSURE

### Technical Problem

Therefore, the present invention has been made in view of the above problems, and it is one object of the present invention to develop and provide a composition that contains 2′-fucosyllactose and is thus highly effective in preventing a stroke.

### Technical Solution

In accordance with the present invention, the above and other objects can be accomplished by the provision of a pharmaceutical composition containing 2′-fucosyllactose (2′-FL) for preventing a stroke.

In the pharmaceutical composition according to the present invention, the stroke is preferably an ischemic stroke.

In accordance with another aspect, provided is a food composition containing 2′-fucosyllactose (2′-FL) for preventing a stroke.

In the food composition according to the present invention, the stroke is preferably an ischemic stroke.

### Advantageous Effects

The pharmaceutical or food composition containing 2′-fucosyllactose according to the present invention is highly effective in preventing a stroke.

### DESCRIPTION OF DRAWINGS

The above and other objects, features and other advantages of the present invention will be more clearly understood from the following detailed description taken in conjunction with the accompanying drawings, in which:

FIG. 1 shows the results of TTC staining of the stroke-induced brain after oral administration of 2′-FL on each week (0-7 weeks) and in each dose (200 mg/kg, 300 mg/kg, 400 mg/kg) (A: image showing stained brain sections, B: graph showing the result of quantification of the damaged part of A); and

FIG. 2 shows the result of measurement of spontaneous exercise (horizontal activity, vertical activity, total travel distance) according to administration of 2′-FL to the stroke-induced animal group.

### BEST MODE

In one aspect, the present invention is directed to a pharmaceutical composition containing 2′-fucosyllactose (2′-FL) for preventing a stroke. Human breast milk contains 200 or more kinds of human milk oligosaccharides (HMOs) having different structures, which are present at a considerably higher concentration (5 to 15 g/L) than other mammals. HMOs provide a variety of biological activities having positive effects on the development and health of infants, such as prebiotic effects that help the growth of intestinal lactic acid bacteria, prevention of pathogen infections, regulation of the immune system and brain development. For this reason, breastfeeding in infancy is known to be very important. HMOs include D-glucose (Glc), D-galactose (Gal), N-acetylglucosamine (GlcNAc), L-fucose (Fuc) and sialic acid [Sia; N-acetyl neuraminic acid (Neu5Ac)].

In the composition according to the present invention, the stroke is preferably an ischemic stroke. Ischemic stroke refers to a condition in which brain tissue cannot perform its functions due to a decrease in cerebral blood flow caused by occlusion of cerebrovascular vessels, and is a term used for both cerebral infarction and transient ischemic attack. According to the the present invention, a composition having an effect of preventing strokes can be developed based on the finding that brain damage in the mouse experimental group administered with 2′-fucosyllactose was significantly suppressed compared to the control group not administered with 2′-fucosyllactose.

Meanwhile, the present invention provides a pharmaceutical composition containing 2′-fucosyllactose for preventing strokes, and the content of 2′-fucosyllactose present in the pharmaceutical composition of the present invention is preferably controlled depending on the method of use of the preventive and therapeutic agent, the conditions of administration, the type of disease and the severity of the disease. The content of 2′-fucosyllactose in the composition of the present invention may be 0.1 to 50% by weight compared to the pharmaceutical composition for preventing strokes, but is not necessarily limited thereto. However, when the content is less than 0.1% by weight, the therapeutic effect may be insignificant, and when the content exceeds 50% by weight, the rate of increase in the effect relative to the amount used may be uneconomically low.

Meanwhile, the pharmaceutical composition for preventing strokes according to the present invention may further contain a pharmaceutically acceptable carrier, diluent or excipient, in addition to the active ingredient. The carrier, excipient or diluent which may be used in the present invention includes lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methylhydroxybenzoate, propylhydroxybenzoate, talc, magnesium stearate and mineral oil, and one or more selected from these substances may be used. In addition, when the preventive and therapeutic agent is a drug, the pharmaceutical composition may further contain a filler, an anticoagulant, a lubricant, a wetting agent, a fragrance, an emulsifier, a preservative or the like.

In addition, the pharmaceutical composition according to the present invention may be formulated into a preferred form depending on the method of use, and in particular, the pharmaceutical composition is preferably formulated by adopting a method well-known in the art to provide rapid, sustained or delayed release of the active ingredient after administration to a mammal. Specific examples of the formulation include any one selected from plasters, granules, liniments, limonades, powders, syrups, liquid formulations, extracts, elixirs, fluidextracts, emulsions, suspensions, decoctions, infusions, tablets, suppositories, injections, spirits, cataplasms, capsules, troches, tinctures, pastes, pills, and soft or hard gelatin capsules.

In addition, the dosage of the pharmaceutical composition for preventing strokes according to the present invention is preferably determined in consideration of factors such as the method of administration, the age, gender and weight of the subject, and the severity of the disease. For example, the pharmaceutical composition for preventing strokes according to the present invention may be administered at least once daily at 0.1 to 100 mg/kg (body weight), based on the active ingredient. However, this dosage is provided only as an example for illustration, and may be changed according to a physician's prescription depending on the condition of the subject.

In another aspect, the present invention is directed to a food composition containing 2′-fucosyllactose (2′-FL) for preventing strokes. In the food composition for preventing strokes according to the present invention, the term “prevention” in the medical sense does not mean absolute prevention of occurrence of a short-term stroke, but means that the occurrence of a stroke can be slowed down or inhibited by ingesting the food composition containing 2′-fucosyllactose for preventing strokes according to the present invention. Therefore, in the food composition of the present invention, “stroke prevention” may mean “delay of stroke occurrence” or “inhibition of stroke occurrence”.

In the food composition containing 2′-fucosyllactose (2′-FL) for preventing strokes, 2′-fucosyllactose is preferably present in an amount of 0.00001 to 50% by weight with respect to the pharmaceutical composition for preventing strokes. When the content is less than 0.00001% by weight, the therapeutic effect may be insignificant, and when the content exceeds 50% by weight, the increase in the effect relative to the amount used may be uneconomically low.

The food composition for preventing strokes according to the present invention includes, for example, any one selected from meat, cereals, caffeinated beverages, general beverages, chocolate, bread, snacks, confectioneries, candy, pizza, jellies, noodles, gum, dairy products, ice cream, alcoholic beverages, liquors, vitamin complexes and other health supplements, but is not necessarily limited thereto.

### MODE FOR INVENTION

Hereinafter, the present invention will be described in more detail with reference to the following examples, but the scope of the present invention is not limited to the examples, and includes variations and technical concepts equivalent thereto.

### Example 1: Determination of Stroke Prevention Effect of 2′-fucosyllactose (2′-FL)

(1) Establishment of Brain-Injury-Induced Model by Middle Cerebral Artery Occlusion (MCAO)

The experimental animals used for stroke induction in this experiment were Sprague-Dawley rats (BioLASCO, Taipei, Taiwan) with an average body weight of 260±20 g, and were divided into control and experimental groups.

In order to establish the brain-injury-induced model through middle cerebral artery occlusion (MCAO), rats were anesthetized with a 10% chloral hydrate solution (administered in 0.4% of body weight), the limbs thereof were fixed, and then a small hole was formed in the right portion of the skull using a drill and the middle cerebral artery (MCA) was tied with 10-0 threads (N-2540, Monosof™ Covidien, Minneapolis, Minn., USA) to block the flow of blood for 1 hour. After 1 hour, blood reperfusion was performed by releasing (removing) the threads blocking the flow of blood. After surgery, the animals were allowed to recover in an incubator maintained at 37° C.

(2) Determination of Stroke Prevention Effect by 2′-FL Using TTC (2,3,5-triphenyltetrazolium Chloride) Staining

In order to determine the effect of preventing strokes by 2′-FL, brain injury was induced in the experimental group administered with 2′-FL on each week (0-7 weeks) and in each dose (200 mg/kg, 300 mg/kg, 400 mg/kg) and in the control group administered with 0.9% saline through middle cerebral artery occlusion (MCAO) as in (1) above. After 1 hour, the brains of the experimental animals (both experimental and control groups) were extracted.

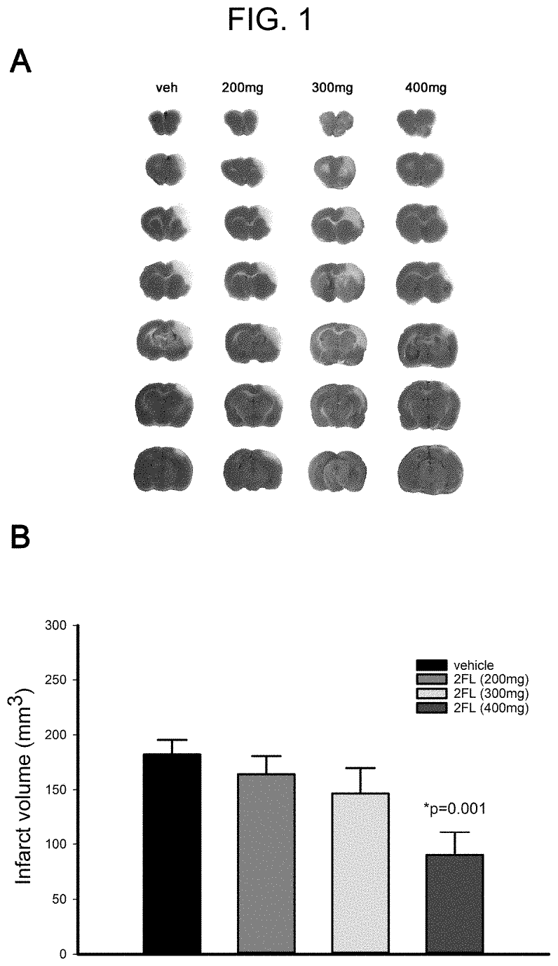

The extracted brains were fragmented using a brain matrix at 2 mm intervals and then stained in a 2% TTC (2,3,5-triphenyltetrazolium chloride) solution for 30 minutes. The stained brain tissue fragments were imaged with a digital camera, and brain damage was measured (FIG. 1). FIG. 1 shows the results of TTC staining of the stroke-induced brain after oral administration of 2′-FL on each week (0-7 weeks) and in each dose (200 mg/kg, 300 mg/kg, 400 mg/kg) (A: image showing stained brain sections, B: graph showing the result of quantification of the damaged part of A).

Upon TTC staining used for experiments, normal cells react with dehydrogenase in the mitochondria and turn red (FIG. 1 is a black-and-white image and thus looks dark), while cerebral infarction cells look white because they are not stained due to lose of cerebral infarction cells, thus being widely used as an indicator of the presence or absence of irreversible damage of cells.

The experimental results showed that the longer the 2′-FL pre-administration period and the higher the dose, the better the effect on brain damage suppression. This demonstrates that the 2′-FL according to the present invention is capable of preventing strokes. That is, the present invention clearly showed the stroke prevention effect of 2′-FL through the brain injury induction model using the middle cerebral artery occlusion (MCAO) of the present invention. In other words, the present invention clearly demonstrates the effect of 2-′FL on prevention of strokes, other than clear demonstration of the effect of 2-′FL on treatment of strokes.

(3) Determination of Stroke Prevention Effect by 2′-FL Using Behavioral Test Analysis

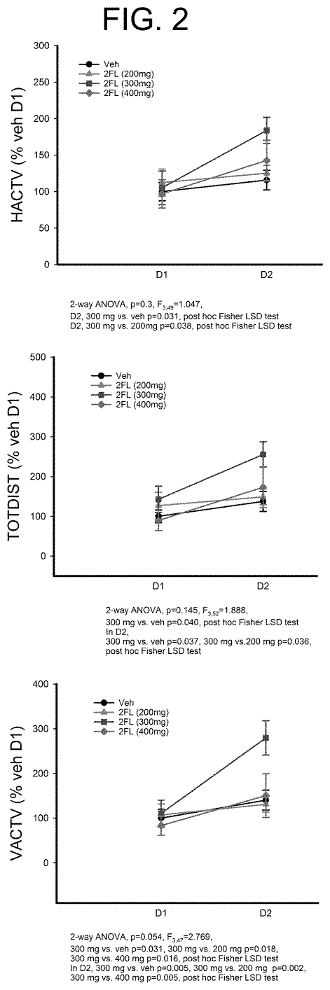

Nerve-damage-induced models such as stroke-induced models tend to decrease spontaneous behavior due to the decline of muscle and nerve functions. In this experiment, to determine the effect of 2′-FL administration on the behavior of stroke-induced animals, 2′-FL was orally administered in different amounts of 200 mg/kg, 300 mg/kg and 400 mg/kg for 3 days, and then brain damage was induced by the method of (1) above. Then, each experimental animal was placed in a transparent box with a size of 42×42×31 cm³ equipped with a beam sensor, and then the quantity of spontaneous exercise (horizontal activity, vertical activity, total distance traveled) was measured on day 1 (D1, one day after stroke induction) and day 2 (D2, two days after stroke induction). FIG. 2 shows the result of measurement of spontaneous exercise (horizontal activity, vertical activity, total travel distance) according to administration of 2′-FL to the stroke-induced animal group.

In FIG. 2, “HACTC” on the vertical axis means horizontal activity, “VACTV” means vertical activity, and “TOTDIST” means total travel distance.

In the group not administered with a drug (veh), neither a significant change in exercise nor improvement in behavior were found two days after stroke induction (D2) compared to one day after stroke induction (D1). Meanwhile, the group pre-administered with 2-FL was found to increase the total exercise amount 2 days after stroke induction (D2), and particularly, the exercise amount was found to significantly increase at 300 mg/kg. At a higher dose of 400 mg/kg, an increase in exercise was found, but there was no statistically significant increase in exercise, unlike the case of a dose of 300 mg/kg. This was considered to be due to the difference in the amount of absorption of 2′-FL with short-term administration.

The behavioral test analysis demonstrated more clearly that 2′-FL of the present invention is capable of preventing strokes.

1. A pharmaceutical composition comprising 2′-fucosyllactose (2′-FL) for preventing a stroke.

2. The pharmaceutical composition according to claim 1, wherein the stroke is an ischemic stroke.

3. A food composition comprising 2′-fucosyllactose (2′-FL) for preventing a stroke.

4. The food composition according to claim 3, wherein the stroke is an ischemic stroke.
